(19) United States
(12) Patent Application Publication
(10) Pub. No.: US 2008/0306565 A1
(43) Pub. Date: Dec. 11, 2008

(54) Medical Device

(76) Inventors: Nils Holmstrom, Jarfalla (SE); Kjell Noren, Solna (SE)

Correspondence Address: SCHIFF HARDIN, LLP;PATENT DEPARTMENT, 6600 SEARS TOWER, CHICAGO, IL, 60606-6473, US

(21) Appl. No.: 12/095,212

(22) PCT Filed: Dec. 16, 2005
(86) PCT No.: PCT/SE05/01957

§ 371 Date: May 28, 2008

(51) Int. Cl. A61N 1/00; A61F 2/02

(52) U.S. Cl. 607/18; 623/11.11; 607/17; 607/30

(57) ABSTRACT

In an implantable medical device and a method and computer-readable medium for operating the medical device to detect a condition of a heart of a patient, activity level of a patient and acoustic energy in a patient are sensed, and acoustic signals are generated that are indicative of heart sounds of the patient over predetermined periods of a cardiac cycle during successive cardiac cycles. A signal corresponding to a first sound is extracted from the sensed acoustic signal of a cardiac cycle. A relation is calculated between a first signal corresponding to the first heart sound in a first activity range, and a second signal corresponding to the first heart sound in a second predetermined activity level range. The calculated relation is compared with at least one reference value to detect the condition or a change of the condition.

### TECHNICAL FIELD

The present invention generally relates to implantable medical devices, such as cardiac pacemakers and implantable cardioverter/defibrillators, and in particular to a method, an implantable medical device, a computer program product and a computer readable medium for detecting a condition of a heart of a patient, such as Congestive Heart Failure (CHF), arrhythmia, conduction disorder, heart insufficiency, increased cardiac output and/or increased stress level.

### BACKGROUND OF THE INVENTION

Auscultation is an important diagnostic method for obtaining information of the heart sounds, which is well established as diagnostic information of the cardiac function. The sounds are often described as S1-S4. During the working cycle of the heart mechanical vibrations are produced in the heart muscle and the major blood vessels. Acceleration and retardation of tissue cause the vibrations when kinetic energy is transformed to sound energy, e.g. at valve closing. Vibrations can also arise from turbulent blood flow, e.g. at stenosis and regurgitation. These vibrations may be listened to using a stethoscope or registered electronically using phonocardiography, i.e. graphical registration of the heart sounds by means of a heart microphone placed on the skin of the patient's thorax. Auscultation using a stethoscope is, to a large extent, built on practical experience and long practice since the technique is based on the doctor's interpretations of the hearing impressions of heart sounds. When applying phonocardiography, as mentioned above, a heart microphone placed on the skin of the patient's thorax. In other words, it may be cumbersome and time-consuming to obtain knowledge of the heart sounds and the mechanical energy during the heart cycle using these manual or partly manual methods and, in addition, the obtained knowledge of the heart sounds may be inexact due to the fact that the knowledge is, at least to some extent, subjective.

The first tone S1 coincides with closure of the Mitral and Tricuspid valves at the beginning of systole. Under certain circumstances, the first tone S1 can be split into two components. An abnormally loud S1 may be found in conditions associated with increased cardiac output (e.g. fever, exercise, hyperthyroidism, and anemia), tachycardia and left ventricular hyperthrophy. A loud S1 is also characteristically heard with mitral stenosis and when the P-R interval of the EKG is short. An abnormally soft S1 may be heard with mitral regurgitation, heart failure and first degree A-V block (prolonged P-R interval). A broad or split S1 is frequently heard along the left lower sternal border. It is a rather normal finding, but a prominent widely split S1 may be associated with right bundle branch block (RBBB). Beat-to-beat variation in the loudness of S1 may occur in atrial fibrillation and third degree A-V block.

The second heart sound S2 coincides with closure of the aortic and pulmonary valves at the end of systole. S2 is normally split into two components (aortic and pulmonary valves at the end of systole) during inspiration. Splitting of S2 in expiration is abnormal. An abnormally loud S2 is commonly associated with systemic and pulmonary hypertension. A soft S2 may be heard in the later stages of aortic or pulmonary stenosis. Reversed S2 splitting (S2 split in expiration—single sound in inspiration) may be heard in some cases of aortic stenosis but is also common in left bundle branch block (LBBB). Wide (persistent) S2 splitting (S2 split during both inspiration and expiration) is associated with right bundle branch block, pulmonary stenosis, pulmonary hypertension, or atrial septal defect.

The third heart sound S3 coincides with rapid ventricular filling in early diastole. The third heart sound S3 may be found normally in children and adolescents. It is considered abnormal over the age of 40 and is associated with conditions in which the ventricular contractile function is depressed (e.g. CHF and cardiomyopathy). It also occurs in those conditions associated with volume overloading and dilation of the ventricles during diastole (e.g. mitral/tricuspid regurgitation or ventricular septal defect). S3 may be heard in the absence of heart disease in conditions associated with increased cardiac output (e.g. fever, anemia, and hyperthyroidism).

The fourth heart sound S4 coincides with atrial contraction in late diastole. S4 is associated with conditions where the ventricles have lost their compliance and have become “stiff”. S4 may be heard during acute myocardial infarction. It is commonly heard in conditions associated with hyperthrophy of the ventricles (e.g. systemic or pulmonary hypertension, aortic or pulmonary stenosis, and some cases of cardiomyopathy). The fourth heart sound S4 may also be heard in patients suffering from CHF.

Thus, the systolic and diastolic heart functions are reflected in the heart sounds. For example, in “A relative value method for measuring and evaluating cardiac reserve”, Xiao S, Guo X, Sun X, Xiao Z, Biomed. Eng. Online, 2002 Dec. 6; 1:6, it was shown that the ratio of S1 amplitude after exercise to that at rest can be used to evaluate cardiac contractility and cardiac reserve mobilization level. It was also shown that the ratio of S1 amplitude to S2 amplitude (S1/S2) may be used as an indicator of hypotension. In “Functional abnormalities in isolated left bundle branch block. The effect of interventricular asynchrony”, Grines C L, Bashore T M, Boudoulas H, Olson S, Shafer P, Wooley C F, Circulation, 1989 April; 79(4):845-53, it was shown that the first heart sound (S1) amplitude, expressed as the ratio S1/S2, was decreased in patients with isolated left bundle branch block (LBBB) due to wide separation of the valvular contributor to S1. As discussed above, an abnormally soft S1 may be heard with mitral regurgitation or heart failure and hence information of the first heart sound S1 may be used to observe the occurrence of or development of heart failure. This would be of great benefit since Congestive Heart Failure (CHF) is a condition that affects thousands of people throughout the world entailing increasing costs for medical services. According to “The Cardio Renal Anemia (CRA) syndrome: Congestive Heart Failure, Chronic kidney insufficiency, and anemia”, Silverberg D. et al., Dialysis Times, Vol. 10, No. 1, September 2004, the prevalence of CHF is about 2% and in those of age 80+ it reaches 10% of the population. The prevalence is rising as people live longer and better survive myocardial infarctions. Knowledge of the heart sounds and the mechanical energy as well as their relations during the heart cycle can thus be used for monitoring or detecting conditions or changes of conditions of a heart of a patient, such as CHF, heart insufficiency, systemic hypertension, or pulmonary hypertension.

However, there is no known technique for continuously and automatically collecting information of the heart sounds, the corresponding energy values, and their relations and for using the information to detect or derive detecting conditions or changes of conditions of a heart of a patient, such as, for example, Congestive Heart Failure, heart insufficiency, systemic hypertension, or pulmonary hypertension.

### BRIEF DESCRIPTION OF THE INVENTION

Thus, an object of the present invention is to provide a method and medical device that are capable of continuously and automatically collecting information of the heart sound and the corresponding energy values and their relations and to use the information to detect or derive conditions or changes of conditions of a heart of a patient using detected and monitored heart sounds and corresponding energy values.

This and other objects are achieved according to the present invention by providing a method, an implantable medical device, a computer program product and a computer readable medium having the features defined in the independent claim. Preferable embodiments of the invention are characterised by the dependent claims.

According to an aspect of the present invention, there is provided an implantable medical device for detecting a condition of a heart of a patient, comprising: an activity sensor adapted to sense an activity level of the patient; a signal processing circuit adapted to extract a signal corresponding to a first heart sound (S1) from a sensed acoustic signal, the signal being received from an acoustic sensor adapted to sense an acoustic energy and to produce acoustic signals indicative of heart sounds of the heart of the patient over predetermined periods of a cardiac cycle during successive cardiac cycles; and a controller adapted to compare a present signal corresponding to a first heart sound (S1) in a first predetermined activity level range with at least one reference value to detect the condition or a change of the condition.

According to a second aspect of the present invention, there is provided a method for detecting a condition of a heart of a patient, comprising the steps of: sensing an activity level of the patient; sensing an acoustic energy; producing acoustic signals indicative of heart sounds of the heart of the patient over predetermined periods of a cardiac cycle during successive cardiac cycles; extracting a signal corresponding to a first heart sound (S1) from a sensed acoustic signal of a cardiac cycle; and comparing a present signal corresponding to a first heart sound (S1) in a first predetermined activity level range with at least one reference value to detect the condition or a change of the condition.

According to a third aspect of the present invention, there is provided a computer program product, which when executed on a computer, performs steps in accordance with the second aspect of the present invention.

According to a further aspect of the present invention, there is provided a computer readable medium comprising instructions for bringing a computer to perform a method according to the second aspect of the present invention.

Thus, the invention is based on the idea of, using an implantable medical device, continuously collecting or obtaining information of heart sound and their relation, which carry valuable information of the workload and status of the heart, and using this information to detect different conditions and changes of such conditions. In particular, the invention is based on the insight that information of the heart sounds and the mechanical energy as well as their relations during the heart cycle can reveal information about Congestive heart Failure (CHF), for example, the severity of CHF. For a healthy person the first heart sound signal (S1) or its power E1 will increase dramatically during exercise due increased contractility. A CHF patient is not capable to increase the contraction, which may be observed by a lack of power increase during exercise. A comparison of a present signal corresponding to a first heart sound (S1) in a first predetermined activity level range, e.g. at rest, with at least one reference value, e.g. an activity value corresponding to exercise, can be used to detect a condition or a change of a condition such as CHF. The reference values may be pre-stored in the device or may include preceding first heart sound signals.

The present invention provides several advantages. For example, one advantage is that the collecting of information and the determining or detecting of conditions or changes of conditions can be performed continuously on an automated basis, which entail that it is possible to study changes of the heart sound and the corresponding energy over time.

Another advantage is that a condition or a changing condition of a heart of patient can be detected or derived at a relatively early stage and in a fast and reliable way since intrinsic information of the heart, i.e. the heart sounds, is used as input information, in turn, leading to a better security for the diagnosis for patients in different situations. The results are also accurate due to the facts that the systolic and diastolic heart functions are reflected in the heart sound, and that the heart sound and their relations thus carry information of the workload and status of the heart.

The fact that the heart sounds may be obtained by means of an implantable medical device connectable to an acoustic sensor that senses sounds or vibrations inside or outside the heart also contributes to higher degree of accuracy and reliability.

A further advantage of the present invention is that it is possible to study changes of the heart sounds and their energy over time, which may provide useful information regarding, for example, the variability of the heart sound and/or the energy parameters. This information can, in turn, be used as an indicator of, for example, a changed filling due to e.g. arrhythmia or conduction disorder. Furthermore, the collected energy information can be used to tune a combination of drugs given to the patient.

In an embodiment of the present invention, a sensing session of the acoustic sensor, i.e. a session for sensing acoustic energy of the heart sounds, is initiated at or synchronized with the detection of a QRS-position, an intrinsic detected event or a paced event. The sensing session may have a predetermined length, for example, a predetermined time window, which window may be programmable and a typical length is about 200 ms.

According to an embodiment of the present invention, a relation between a first signal corresponding to a first heart sound (S1) in the first activity level range and a second signal corresponding to a first heart sound (S1) in a second predetermined activity level range is calculated and compared with at least one reference value to detect the condition or a change of the condition. For example, the maximum amplitudes of the first heart sounds in the two different activity level ranges are used. The reference values may be pre-stored in the device or may be preceding first heart sound signals and/or energy values.

In an embodiment of the present invention, an energy value corresponding to an extracted signal corresponding to a first heart sound (S1) in a first activity level range is calculated and compared with at least one reference value to detect the condition or a change of the condition.

According to a further embodiment of the present invention, the relation is calculated as a relation between an energy value corresponding to a first heart sound at the first activity level range and an energy value corresponding to a first heart sound at the second activity level range and compared with at least one reference value to detect the condition or a change of the condition.

In a further embodiment of the present invention, a calculated relation between an energy value corresponding to a first heart sound at the first activity level range and an energy value corresponding to a first heart sound at the second activity level range is compared with at least one reference value to detect the condition or the change of said condition, wherein a determination that the calculated relation is within a predetermined reference value range, i.e. lower than a predetermined first reference value or higher than a second reference value, indicates the occurrence of the condition. That is, the conditions is detected if the energy value does not increase with a predetermined factor when the activity is increased from a first activity level to a second activity level. For example, factors related to different activity level relations may be pre-stored in the device.

According to yet another embodiments of the present invention, each reference value is calculated as a mean value of a predetermined number of signals corresponding to preceding first heart sounds or as mean value of a predetermined number of energy values corresponding to preceding first heart sounds. Thereby, more reliable and accurate energy values and/or heart sound signals can be obtained. Alternatively, a weighted average value of a predetermined number of successive energy values or heart sound signals can be used. In still another embodiment, a moving average of a predetermined number of successive energy values or heart sound signals is utilized.

In further embodiments of the present invention, each signal corresponding to a first heart sound (S1) at the first activity level is calculated as a mean value over a predetermined number of successive heart sound signals at the first activity level and each signal corresponding to a first heart sound (S1) at the second activity level is calculated as a mean value over a predetermined number of successive heart sound signals at the second activity level. Further, each energy value corresponding to a first heart sound at the first activity level can be calculated as a mean value over a predetermined number of successive energy values at the first activity level and each energy value corresponding to a first heart sound at the second activity level is calculated as a mean value over a predetermined number of successive energy values at the second activity level.

According to embodiments of the present invention, conditions including Congestive Heart Failure (CHF), arrhythmia, conduction disorder, heart insufficiency, increased cardiac output and/or increased stress level may be detected or derived. By continuously obtaining energy values corresponding to the first heart sound it is possible to study changes of the energy E1 over time and thereby changes and/or prevalence of such conditions. For example, a high variance of the energy parameters during otherwise constant conditions, e.g. at rest, indicates that filling is altering due to e.g. arrhythmia or conduction disorder. If E1 becomes low value may be an indication of heart insufficiency and if E1 becomes high it may be an indication of stress or increased cardiac output.

In yet another embodiment of the present invention, a bandpass filter is adapted to filter off frequency components of the acoustic signal outside a predetermined frequency range. The bandpass filter may have a frequency range of 10 to 300 Hz. The filtered signal is rectified to produce at least one signal containing only positive or zero values and at least one local maximum point being coincident with a first heart sound signal and at least one local maximum point being coincident with a second heart sound signal that are identified in the rectified signal. To produce the energy values corresponding to the first heart sound, the sound signal can be integrated in a predetermined time window comprising the at least one local maximum point. Alternatively, a squaring procedure is performed on the filtered signal to produce at least one signal containing only positive or zero values. At least one local maximum point being coincident with a first heart sound signal is identified in the squared signal. To produce the energy values corresponding to the first heart sound, the sound signal can be integrated in a predetermined time window comprising the at least one local maximum point.

In a further embodiment of the present invention, a part of the signal containing only positive or zero values, i.e. the rectified or squared signal, above a predetermined threshold is selected and the part of the signal above the predetermined threshold is integrated, wherein an energy value corresponding to the first heart sound can be obtained.

In an alternative embodiment of the present invention, at least one body position of the patient is detected and it is determined whether the patient is in at least one predetermined specific body position. In one embodiment of the present invention, the position detecting means is a back-position sensor arranged to sense when the patient is lying on his/hers back (or on his or hers face). The position information may be used in the detection or deriving of the condition or change of the condition. Moreover, a sensing session of the acoustic sensor may be synchronized with a determination that the patient is in a predetermined position. Thereby, it is possible to obtain the heart sound signals at stable or similar conditions, which, in turn, entails more reliable and accurate results with respect to, for example, the detection of a condition. Of course, one or more positions can be detected, for example, when the patient is in supine (lying down) and when the patient is in an upright position and sensing sessions of the acoustic sensor may be synchronized with these positions.

In yet another embodiment of the present invention, a heart rate of the patient is sensed and it is determined or checked whether a sensed heart rate is within a predetermined heart rate interval. The heart rate information can be used in the detection or deriving of a condition or change of a condition. A sensing session of the acoustic sensor can be synchronized with a determination that the sensed heart rate level is within a predetermined heart rate level range, below a predetermined heart rate level or above a predetermined heart rate level. Thereby, it is possible to obtain the heart sound signals at stable or similar conditions, which, in turn, entails more reliable and accurate results with respect to, for example, the detection of a condition.

In embodiments, the acoustic sensor is arranged in a lead connectable to the device and is located e.g. in the right ventricle of the heart of the patient, in the left ventricle or in a coronary vein on the middle of the left ventricle. In general, the sensor can be positioned anywhere in thorax.

According to embodiments, the acoustic sensor is an accelerometer, a pressure sensor or a microphone.

In an alternative embodiment of the present invention, the sensor is arranged within a housing of the implantable device.

As realized by the person skilled in the art, the methods of the present invention, as well as preferred embodiments thereof, are suitable to realize as a computer program or a computer readable medium.

The features that characterize the invention, both as to organization and to method of operation, together with further objects and advantages thereof, will be better understood from the following description used in conjunction with the accompanying drawings. It is to be expressly understood that the drawings is for the purpose of illustration and description and is not intended as a definition of the limits of the invention. These and other objects attained, and advantages offered, by the present invention will become more fully apparent as the description that now follows is read in conjunction with the accompanying drawings.

### BRIEF DESCRIPTION OF THE DRAWINGS

In the following detailed description, reference will be made to the accompanying drawings, of which:

FIG. 1 is schematic diagram showing a medical device implanted in a patient in which device the present invention can be implemented.

FIG. 2 is block diagram of the primary functional components of a first embodiment of the medical device according to the present invention.

FIGS. 3a and 3b are block diagrams of embodiments of a signal processing circuit according to the present invention.

FIG. 4 is a block diagram of the primary functional components of another embodiment of the medical device according to the present invention.

FIG. 5a is a flow chart of an embodiment of the method according to the present invention.

FIG. 5b is a flow chart of another embodiment of the method according to the present invention.

FIG. 6 shows a typical cardiac cycle, related heart sounds, and the resulting signals at a heart rate of 75 BPM.

FIG. 7 shows signal processing steps performed in accordance with an embodiment of the method according to the present invention.

### DETAILED DESCRIPTION OF THE INVENTION

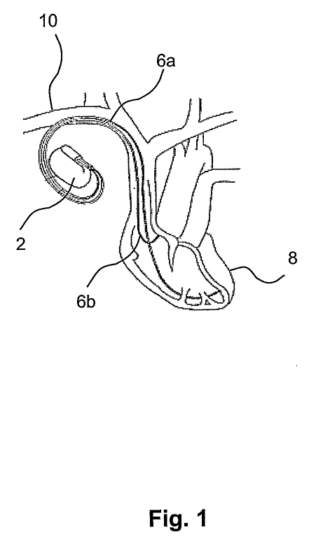

With reference to FIG. 1, there is shown a schematic diagram of a medical device implanted in a patient in which device the present invention can be implemented. As seen, this embodiment of the present invention is shown in the context of a pacemaker 2 implanted in a patient (not shown). The pacemaker 2 comprises a housing being hermetically sealed and biologically inert. Normally, the housing is conductive and may, thus, serve as an electrode. The pacemaker 2 is connectable to one or more pacemaker leads, where only two are shown in FIG. 1 namely a ventricular lead 6a and an atrial lead 6b. The leads 6a and 6b can be electrically coupled to the pacemaker 2 in a conventional manner. The leads 6a, 6b extend into the heart 8 via a vein 10 of the patient. One or more conductive electrodes for receiving electrical cardiac signals and/or for delivering electrical pacing to the heart 8 are arranged near the distal ends of the leads 6a, 6b. As the skilled man in the art realizes, the leads 6a, 6b may be implanted with its distal end located in either the atrium or ventricle of the heart 8, or in the coronary sinus or in the cardiac vein or in a coronary vein on the left ventricle, or they may be in form of epicardial leads attached directly at the epicardium.

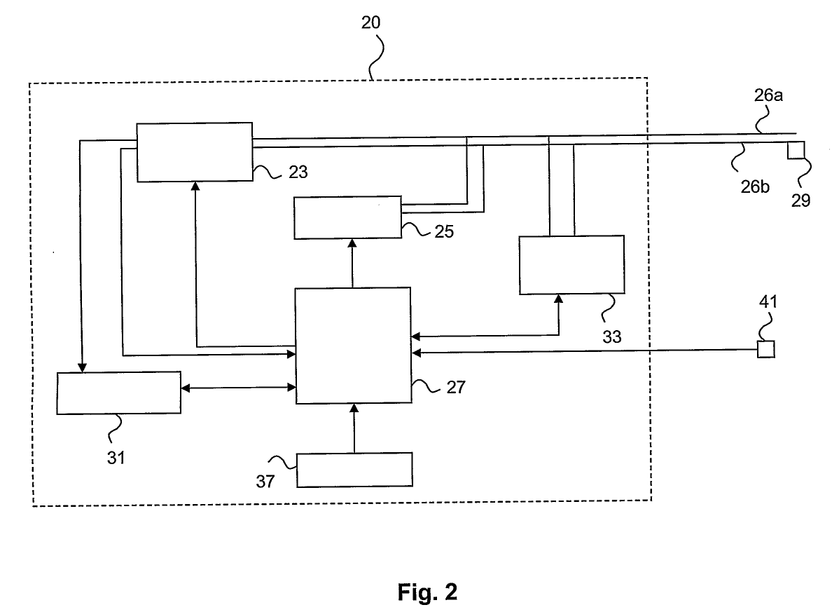

With reference now to FIG. 2, the configuration including the primary components of an embodiment of the present invention will be described. The illustrated embodiment comprises an implantable medical device 20, such as the pacemaker shown in FIG. 1. Leads 26a and 26b, of the same type as the leads 6a and 6b shown in FIG. 1, are connectable to the device 20. The leads 26a, 26b may be unipolar or bipolar, and may include any of the passive or active fixation means known in the art for fixation of the lead to the cardiac tissue. As an example, the lead distal tip (not shown) may include a tined tip or a fixation helix. The leads 26a, 26b comprises one or more electrodes (as described with reference to FIG. 1), such as a tip electrode or a ring electrode, arranged to, inter alia, measure the impedance or transmit pacing pulses for causing depolarization of cardiac tissue adjacent to the electrode(-s) generated by a pace pulse generator 25 under influence of a controller 27 including a microprocessor. The controller 27 controls, inter alia, pace pulse parameters such as output voltage and pulse duration.

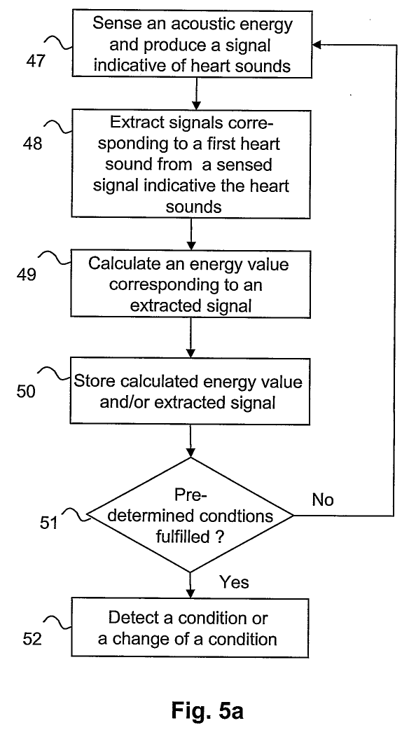

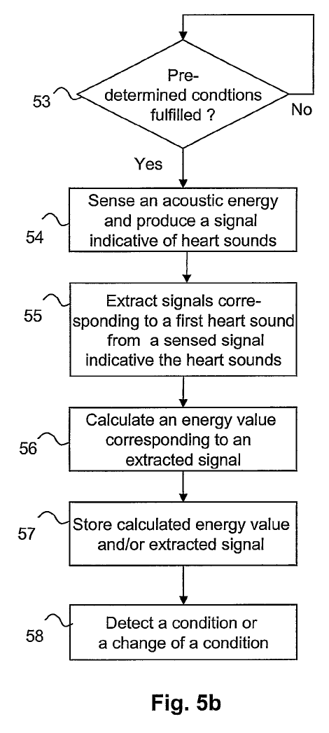

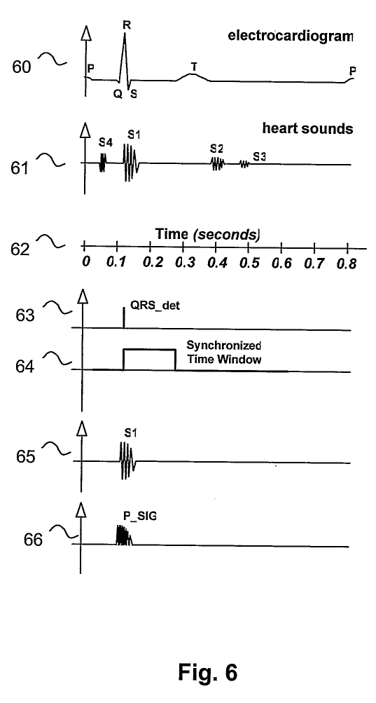

Furthermore, an acoustic sensor 29 is arranged in or connected to one of the leads 26a, 26b. Alternatively, the acoustic sensor is located within the housing of the device 20. In one embodiment, the acoustic sensor 29 is arranged in a lead located in a right ventricle of the heart, in coronary sinus or in a cardiac vein of the patient. The acoustic sensor 29 may, for example, be an accelerometer, a pressure sensor, or a microphone. The acoustic sensor 29 is adapted to sense acoustic energy of the heart and to produce signals indicative of heart sounds of the heart of the patient. For example, the acoustic sensor 29 may sense the acoustic energy over predetermined periods of a cardiac cycle during successive cardiac cycles. In one embodiment of the present invention, a sensing session to obtain a signal indicative of a first heart sound (S1) is synchronized with a detected heart event, e.g. detection of an intrinsic or paced QRS-complex, which will be discussed in more detail hereinafter with reference to FIGS. 5a, 5b, and 6.

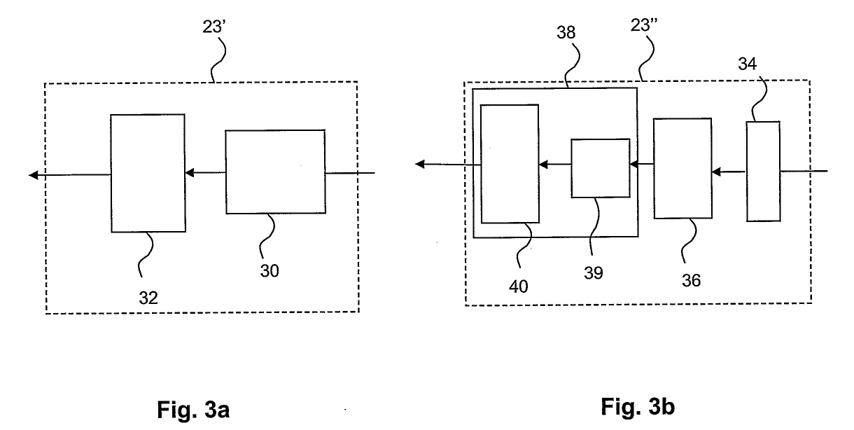

Furthermore, the implantable medical device 20 comprises a signal processing circuit 23 adapted to process the sensed signal to extract a signal corresponding to a first heart sound (S1) and to calculate an energy value (E1) corresponding to the extracted signal, which will be described in further detail hereinafter with reference to FIGS. 3a and 3b.

A storage means 31 is connected to the controller 27, which storage means 31 may include a random access memory (RAM) and/or a non-volatile memory such as a read-only memory (ROM). Detected signals from the patients heart are processed in an input circuit 33 and are forwarded to the controller 27 for use in logic timing determination in known manner. The implantable medical device 20 is powered by a battery 37, which supplies electrical power to all electrical active components of the medical device 20. Data contained in the storage means 31 can be transferred to a programmer (not shown) via a programmer interface (not shown) for use in analyzing system conditions, patient information, etc.

Furthermore, the implantable medical device includes an activity level sensor 41 for sensing an activity level of the patient connected to the controller 27. The controller 27 may be adapted to determine whether a sensed activity level is within predetermined activity level ranges and/or a below or above a predetermined activity level. These ranges and/or levels may be programmed into the memory 31, for example, at the time of implant by the physician, and they can also be re-programmed using a programmer (not shown) via a programmer interface (not shown). The controller 27 may be adapted to synchronize a sensing session of the acoustic sensor 29 with a determination that the sensed activity level is below a predetermined activity level or that the sensed activity level is within a activity level range between a second activity level and a third activity level.

With reference now to FIGS. 3a and 3b, embodiments of the signal processing circuit will be described. According to one embodiment, see FIG. 3a, the signal processing circuit 23′ comprises a amplitude threshold comparator circuit 30 adapted to determine signals corresponding to a first heart sound (S1) of a sensed signal to be parts of the sensed signal having an amplitude above a predetermined amplitude level. The circuit 23′ also comprises an integrator 32 adapted to integrate a sensed signal during a predetermined time window to calculate an energy value corresponding to the first heart sound signal.

In yet another embodiment, see FIG. 3b, the signal processing circuit 23′ comprises pre-process circuits including one bandpass filter 34 adapted to filter off frequency components of the sensed signals outside a predetermined frequency range and a determining circuit 36 adapted to determine the absolute value of the bandpass filtered signal and to produce a resulting absolute value heart sound signal. The bandpass filter 34 may be a digital filter of second order and adapted to perform a zero-phase procedure to cancel out time delays introduced by the filter. The bandpass filter 34 may be adapted to cut out a predetermined frequency range corresponding to typical frequencies for the first and second heart sound, for example, 10-300 Hz. The bandpass filtered signal is supplied to the absolute value determining means 36 adapted to determine an absolute value (or values) of the incoming signal (or signals) to produce a signal or signals having only positive or zero values. As alternatives to the determining means, a rectifier can be used to rectify the filtered signal or the filtered signal can be squared to obtain the instantaneous power of the filtered signal. The signal processing circuit 23″ also comprises an energy calculating circuit 38 adapted to calculate an energy value corresponding to the filtered signal. For example, the energy calculating circuit 38 may include an identifying circuit 39 adapted to identify at least one local maximum point being coincident with a first heart sound (S1) and an integrator 40 adapted to integrate the signal over a predetermined time window comprising the local maximum points, wherein an energy value of the filtered signal can be obtained.

The medical device 20 according to the present invention may also comprise alarm means (not shown) adapted to send alarm signal indicating that a specific condition has been detected or if a change of a specific condition has been detected. That is, the controller sends a triggering command to the alarm means if a specific condition has been detected or if a change of a specific condition has been detected. The alarm means may be a vibrator causing the device to vibrate or it may be adapted to deliver a beeping sound in order to alert the patient of the situation. Furthermore, an alarm signal can, for example, also or instead be sent to the programmer (not shown) via the programmer interface (not shown). In one embodiment, the alarm means is integrated into the controller 27.

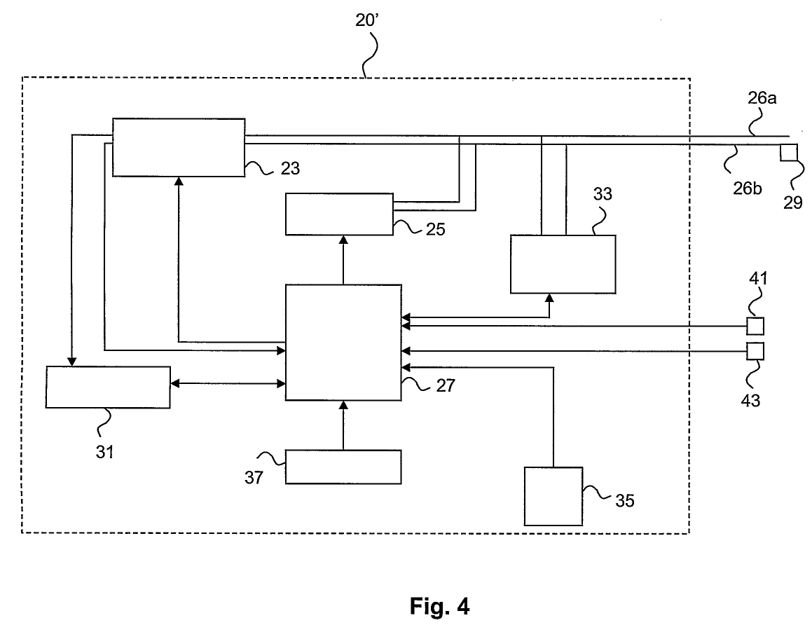

With reference now to FIG. 4, another embodiment of the present invention will be described. Like parts in FIG. 2 and FIG. 3 are denoted with the same reference numeral and the description thereof will be omitted since they have been described with reference to FIG. 2. The implantable medical device 20′ may include a position detecting sensor 35 arranged to detect a body position of the patient. For example, the position sensor 35 can adapted to detect a predetermined specific body position. In a one embodiment of the present invention, the position detecting means is a back-position sensor arranged to sense when the patient is lying on his/hers back (or on his or hers face). The position detecting sensor 35 is connected to the controller 27. The controller 27 may be adapted to determine whether the patient is in the at least one predetermined specific body position and to use the position information in the detection of the condition or change of the condition. In another embodiment, the controller 27 is adapted to synchronize a sensing session of the acoustic sensor 29 with a determination that the patient is in a predetermined position. The position detecting sensor 35 may be adapted to detect two or more predetermined specific body positions and the controller 27 may be adapted to synchronize a sensing session of the acoustic sensor 29 with a determination that the patient is in one of these predetermined positions.

Further, the implantable medical device 20′ according to the present invention may include a heart rate sensor 43 for sensing a heart rate of the patient connected to the controller 27. The controller 27 may be adapted to determine whether a sensed heart rate is within a predetermined heart rate interval and to the controller is adapted to determine whether the heart rate is within a predetermined heart rate level range and to use the heart rate information in the detection of the condition or change of the condition. Moreover, the controller 27 may be adapted to synchronize a sensing session of the acoustic sensor 29 with a determination that the sensed heart rate level is within a predetermined heart rate level range, below a predetermined heart rate level or above a predetermined heart rate level.

As the skilled man realizes, only one, some of or all of the following features: the activity level sensing means 41, the heart rate sensor 43, or the position detector 35 may be included in the medical device according to the present invention. Thus, information from one, some of, or all of the above-mentioned sensors can be used in the detection.

Turning now to FIGS. 5a and 5b, high-level descriptions of the method according embodiments of the present invention will be given. With reference first to FIG. 5a, a first embodiment will be described. First, at step 47, the acoustic sensor 29 senses an acoustic energy and produces signals indicative of heart sounds of the heart of the patient. This may be performed over predetermined periods of a cardiac cycle during successive cardiac cycles under control of the controller 27. The sensor 29 can be adapted to sense the acoustic energy during predefined time windows in the heart cycle. In FIG. 6, a typical cardiac cycle, related heart sounds, and the resulting signals at a heart rate of 75 BPM are shown. A surface electrocardiogram and the related heart sounds S1, S2, S3, and S4 are indicated by 60 and 61, respectively, and a time axis is indicated by 62. In one embodiment, the acoustic sensor 29 is activated by the detection of a QRS-position, as indicated by 63, an intrinsic detected event or a paced event indicated by 60. The acoustic sensor 29 senses the acoustic energy in the heart sound S1, indicated by 62, during a sensing session having a predetermined length, for example, during a predetermined time window, indicated by 64. In this embodiment, the initiation of the sensing session is synchronized with the detection of the QRS-position. The length of the time window is programmable and a typical length is about 200 ms. Hence, the acoustic sensor 29 receives a triggering signal from the controller 27 upon detection of the QRS-position by the input circuit 33. The produced signal corresponding to the first heart sound S1 is indicated by 65. This may be performed during successive cardiac cycles under control of the controller 27, which thus produces a time series of successive heart sound signals. The produced signal or signals indicative of the first heart sounds are supplied to the signal processing circuit 23 where, at step 48, a signal corresponding to a first heart sound (S1) is extracted from a sensed signal by the pre-processing circuits 30, 32, or 34, 36. Optionally, this step may include performing a filtering procedure in order to filter the sensed signal. In one embodiment, frequency components of the signal outside a predetermined frequency range is filtered off and the absolute value of the sensed signal is calculated. The resulting signal is indicated by 66 in FIG. 5. In another embodiment, the first heart sound signal is determined to be a part of the sensed signal having an amplitude above a predetermined amplitude level.

Optionally, this step may include performing a filtering procedure in order to filter the sensed signal. In one embodiment, a first heart sound signal is determined to be a part of the sensed signal having an amplitude above a predetermined amplitude level. Then, in order to calculate the energy value E1, the first heart sound signal is integrated, for example, over a predetermined time window. Alternatively, the sensed signal can be integrated over a first time window to calculate an energy value (E1) corresponding to the first heart sound, and in this case the filtering procedure is thus not necessary.

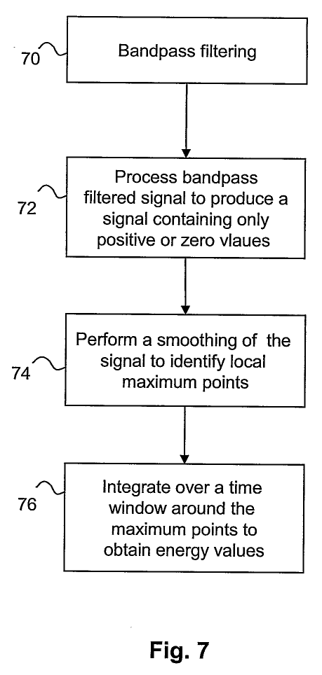

Another embodiment of the filtering procedure will now be discussed with reference to FIGS. 3b and 7. First, at step 70, the sensed signal is bandpass filtered. The bandpass filter 34 is adapted to receive a heart sound signal waveform comprising the first heart sound signal, see 65 in FIG. 6, and to cut out a frequency range of 10-300 Hz to form a signal containing the first heart sound (S1), see signal waveform 66 in FIG. 6a. The bandpass filtering process are performed as a zero-phase procedure to cancel out time delays introduced by the filters. The sensed signal is in fact filtered twice, first in the forward direction and second in the backward direction. Then, at step 72, the bandpass filtered signals are processed to obtain a signal containing only positive or zero values, for example, in the absolute value determining means 36 or a rectifier. Alternatively, the signals can be squared in a squaring means resulting in signal corresponding to the instantaneous power of the bandpass filtered signals. Thereafter, at step 74, a smoothing procedure of the signals is performed in the identifying means 38 to identify at least one local maximum point being coincident with a first heart sound signal and at least one local maximum point being coincident with a second heart sound signal. The smoothed signals are then supplied to the integrator 40 where, at step 76, the first heart sound signal is integrated over a predetermined time window comprising the at least one local maximum point to calculate an energy value (E1) corresponding to the first heart sound.

Returning now to FIG. 5, an energy value corresponding to the extracted signal, indicated by 66 in FIG. 5, corresponding to the first heart sound is calculated, at step 49, in the energy calculating circuit 38. Then, at step 50, the calculated energy values and/or the extracted signal may be stored in the memory means 31. If signals corresponding to the first heart sound is obtained for successive cardiac cycles, the signals and calculated energy values can be stored in the memory means 31 in consecutive time order. As an alternative, the first heart sound signal may be stored after step 48 and before the calculation step 49, and the calculate energy values may be stored after step 49.

Subsequently, at step 51, it is checked whether at least one predetermined condition is fulfilled including a check whether a sensed activity level of the patient is in a predetermined activity level range. If no, the procedure returns to step 47. On the other hand, if yes, the procedure proceeds to step 52, where a procedure to detect or derive a condition or a change of a condition, such as Congestive Heart Failure (CHF), arrhythmia, conduction disorder, heart insufficiency, increased cardiac output and/or increased stress level, is performed by comparing the heart sound signal (signals) or the corresponding energy value (values) with at least one reference value. According to an embodiment of the present invention, a relation between a first signal corresponding to a first heart sound (S1) in the first activity level range and a second signal corresponding to a first heart sound (S1) in a second predetermined activity level range is calculated and compared with at least one reference value to detect the condition or a change of the condition. For example, the maximum amplitudes of the first heart sounds in the two different activity level ranges are used. The reference values may be pre-stored in the device or may be preceding first heart sound signals and/or preceding energy values. They may be programmed into the memory 31, for example, at the time of implant by the physician, and they can also be re-programmed using a programmer (not shown) via a programmer interface (not shown).

In an embodiment of the present invention, an energy value corresponding to an extracted signal corresponding to a first heart sound (S1) in a first activity level range is calculated and compared with at least one reference value to detect the condition or a change of the condition. According to a further embodiment of the present invention, the relation is calculated as a relation between an energy value corresponding to a first heart sound at the first activity level range and an energy value corresponding to a first heart sound at the second activity level range and compared with at least one reference value to detect the condition or a change of the condition. According to yet another embodiments of the present invention, each reference value is calculated as a mean value of a predetermined number of signals corresponding to preceding first heart sounds or as mean value of a predetermined number of energy values corresponding to preceding first heart sounds. Alternatively, a weighted average value of a predetermined number of successive energy values or heart sound signals can be used. In still another embodiment, a moving average of a predetermined number of successive energy values or heart sound signals is utilized. In further embodiments of the present invention, each signal corresponding to a first heart sound (S1) at the first activity level is calculated as a mean value over a predetermined number of successive heart sound signals at the first activity level and each signal corresponding to a first heart sound (S1) at the second activity level is calculated as a mean value over a predetermined number of successive heart sound signals at the second activity level. Further, each energy value corresponding to a first heart sound at the first activity level can be calculated as a mean value over a predetermined number of successive energy values at the first activity level and each energy value corresponding to a first heart sound at the second activity level is calculated as a mean value over a predetermined number of successive energy values at the second activity level.

By continuously obtaining energy values corresponding to the first heart sound it is possible to study changes of the energy E1 over time and thereby also changes and/or prevalence of the above mentioned conditions. For example, a high variance of the energy parameters during otherwise constant conditions, e.g. at rest, indicates that filling is altering due to e.g. arrhythmia or conduction disorder. If E1 becomes low value may be an indication of heart insufficiency and if E1 becomes high it may be an indication of stress or increased cardiac output. The predetermined activity level range can, for example, be set to a range corresponding to a rest level of the patient. Moreover, a CHF patient is not capable to increase the contraction, which may be observed by a lack of power increase during exercise, i.e. the energy of S1 does not increase during exercise. A comparison of a present signal corresponding to a first heart sound (S1) in a first predetermined activity level range, e.g. at rest, with at least one reference value, e.g. an activity value corresponding to exercise, can be used to detect a condition or a change of a condition such as CHF.

Turning now to FIG. 5b, a second embodiment will be described. First, at step 53, it is checked whether at least one predetermined condition is fulfilled including a check whether a sensed activity level of the patient is in a predetermined activity level range. It may also be checked whether other conditions are fulfilled, such as whether the patient is in a predetermined position. If it is determined that the predetermined condition or conditions are not fulfilled, the procedure is repeated. On the other hand, if yes, the procedure proceeds to step 54, where a sensing session of the acoustic sensor 29 is initiated, i.e. the acoustic sensor senses an acoustic energy and produces signals indicative of heart sounds of the heart of the patient. This may be performed over predetermined periods of a cardiac cycle during successive cardiac cycles under control of the controller 27. For example, a sensing session to obtain a signal indicative of a first heart sound (S1) is synchronized with a detected heart event, e.g. detection of an intrinsic or paced QRS-complex as described above with reference to FIG. 6. That is, according to this embodiment, a sensing session is initiated when a sensed activity level of the patient is in the predetermined activity level range and at a detection of an intrinsic or paced QRS-complex. Of course, as the skilled man realizes, there are a number of conceivable alternatives for performing the sensing of the heart sounds. For example, a check whether the patient is in a predetermined position may also be performed.

Thereafter, at step 55, a signal corresponding to a first heart sound (S1) is extracted from the sensed signal by the preprocessing circuits 30, 32, or 34, 36. This may be performed in accordance with the description above with reference to step 48. Then, at step 56, an energy value corresponding to the extracted signal, indicated by 66 in FIG. 5, corresponding to the first heart sound is calculated in the energy calculating circuit 38. This may be performed in accordance with the description above with reference to step 49. Then, at step 57, the calculated energy values and/or the extracted signal may be stored in the memory means 31. If signals corresponding to the first heart sound is obtained for successive cardiac cycles, the signals and calculated energy values can be stored in the memory means 31 in consecutive time order. This may be performed in accordance with the description above with reference to step 50. Finally, at step 58, a procedure to detect or derive a condition or a change of a condition, such as Congestive Heart Failure (CHF), arrhythmia, conduction disorder, heart insufficiency, increased cardiac output and/or increased stress level, is performed by comparing the heart sound signal (signals) or the corresponding energy value (values) with at least one reference value. This may be performed in accordance with the description above with reference to step 52.

As the man skilled within the art realizes, certain steps discussed above must not be performed in the order described above. For example, the step of storing extracted signals can be performed partly before step 49 and 55, respectively, i.e. the extracted signals can be stored before steps 49 and 55, respectively, is executed and the energy values can be stored after steps 49 and 56, respectively, is executed.

Although an exemplary embodiment of the present invention has been shown and described, it will be apparent to those having ordinary skill in the art that a number of changes, modifications, or alterations to the inventions as described herein may be made. Thus, it is to be understood that the above description of the invention and the accompanying drawings is to be regarded as a non-limiting example thereof and that the scope of protection is defined by the appended patent claims.

We claim as our invention:

1. An implantable medical device for detecting a condition of a heart of a patient, comprising:
an activity sensor adapted to sense an activity level of said patient;
a signal processing circuit configured to extract a signal corresponding to a first heart sound (S1) from a sensed acoustic signal, said signal being received from an acoustic sensor adapted to sense an acoustic energy and to produce acoustic signals indicative of heart sounds of the heart of said patient over predetermined periods of a cardiac cycle during successive cardiac cycles; and
a controller configured to calculate a relation between a first signal corresponding to a first heart sound (S1) in said first activity level range and a second signal corresponding to a first heart sound (S1) in a second predetermined activity level range, and to compare said calculated relation with at least one reference value to detect said condition or a change of said condition.

2. (canceled)

3. The implantable medical device according to claim 1, wherein
said signal processing circuit is configured to calculate an energy value corresponding to an extracted signal corresponding to a first heart sound (S1); and wherein
said controller is configured to compare a present energy value corresponding to a first heart sound (S1) in a first activity level range with at least one reference value to detect said condition or a change of said condition.

4. The implantable medical device according to claim 4, wherein
said controller is configured to calculate said relation as a relation between an energy value corresponding to a first heart sound at said first activity level range and an energy value corresponding to a first heart sound at said second activity level range, and to compare said calculated relation with at least one reference value to detect said condition or a change of said condition.

5. The implantable medical device according to claim 3, wherein
said controller is configured to compare said calculated relation between an energy value corresponding to a first heart sound at said first activity level range and an energy value corresponding to a first heart sound at said second activity level range with at least one reference value to detect said condition or a change of said condition, wherein said condition is detected if the calculated relation is lower than a first predetermined reference value or higher than a second predetermined reference value.

6. The implantable medical device according to claim 1, further comprising a storage unit that stores signals and/or energy values corresponding to first heart sounds in said first and/or said second activity level.

7. The implantable medical device according to claim 1 comprising employing, as said at least one reference value, an extracted signal corresponding to a preceding first heart sound or an energy value corresponding to a preceding first heart sound.

8. The implantable medical device according to claim 1, wherein said controller is configured to calculate said at least one reference value as a mean value of a predetermined number of signals corresponding to preceding first heart sounds or as mean value of a predetermined number of energy values corresponding to preceding first heart sounds.

9. The implantable medical device according to claim 7, wherein said controller is configured to compare a present signal corresponding to a first heart sound or a relation with a predetermined number of reference values or with reference values obtained during a predetermined period of time.

10. The implantable medical device according to claim 1, wherein said controller is configured to calculate each signal corresponding to a first heart sound (S1) at said first activity level as a mean value over a predetermined number of successive heart sound signals at said first activity level and each signal corresponding to a first heart sound (S1) at said second activity level as a mean value over a predetermined number of successive heart sound signals at said second activity level.

11. The implantable medical device according to claim 4, wherein said controller is configured to calculate each energy value corresponding to a first heart sound at said first activity level as a mean value over a predetermined number of successive energy values at said first activity level and an energy value corresponding to a first heart sound at said second activity level as a mean value over a predetermined number of successive energy values at said second activity level.

12. The implantable medical device according to claim 1, wherein said signal processing circuit is configured to determine said first heart sound signal to be a part of said sensed signal having an amplitude above a predetermined amplitude level.

13. The implantable medical device according to claim 1, wherein said signal processing circuit comprises an integrator adapted to integrate a sensed signal during a first predetermined time window to obtain an energy value corresponding to said first heart sound signal.

14. The implantable medial device according to claim 1, wherein said signal processing circuit comprises a bandpass filter that filters out frequency components of said acoustic signals outside a predetermined frequency range; and
a rectifier that rectifies said filtered signal to produce signals containing only positive or zero values.

15. The implantable medial device according to any one of preceding claims 1, wherein said signal processing circuit comprises
bandpass filter that filters out frequency components of said sensed signals outside a predetermined frequency range; and
a squaring circuit that squares said filtered signal to produce a signal containing only positive or zero values.

16. The implantable medical device according to claim 14, wherein said signal processing circuit comprises:
an identifying unit that identifies at least one local maximum point being coincident with a first heart sound signal and at least one local maximum point being coincident with a second heart sound signal; and
an integrator that integrates said first heart sound signal in a predetermined time window comprising said at least one local maximum point.

17. The implantable medical device according to claim 14, wherein said signal processing circuit comprises:
an amplitude threshold selector that selects a part of said signal
containing only positive or zero values above a predetermined threshold; and
an integrator that integrates the part of the signal above said threshold, wherein an energy value corresponding to the first heart sound is obtained.

18. The implantable medical device according to claim 1, wherein controller is configured to detect said condition or said change of said condition selected from the group consisting of arrhythmia, a conduction disorder, heart insufficiency, stress, and CHF.

19. The implantable medical device according to claim 1, further comprising
a position detector that detects at least one body position of said patient; and wherein said controller is configured to determine whether said patient is in said at least one predetermined specific body position and to use the position information in said detection of said condition or change of said condition.

20. The implantable medical device according to claim 19, wherein said controller is configured to synchronize a sensing session of said acoustic sensor with a determination that said patient is in a predetermined position.

21. The implantable medical device according to claim 1, wherein said controller is adapted to synchronize a sensing session of said acoustic sensor with a determination that said sensed activity level is below said predetermined activity level an/or that said sensed activity level is within a activity level range between a second activity level and a third activity level.

22. The implantable medical device according to claim 1 further comprising
a heart rate sensor that senses a heart rate of said patient; and wherein
said controller is configured to determine whether said heart rate is within a predetermined heart rate interval and to use said heart rate information in said detection of said condition or change of said condition.

23. The implantable medical device according to claim 1, wherein said acoustic sensor is arranged in a lead connectable to said device.

24. The implantable medical device according to claim 1, wherein said acoustic sensor is arranged within a housing of said device.

25. The implantable medical device according to claim 23, wherein said acoustic sensor is located in a lead configured for placement in a left ventricle, in a right ventricle, or in a coronary vein of said patient.

26. The implantable medical device according to claim 1, wherein said acoustic sensor is selected from the group consisting of acceleration, pressure sensors and microphones.

27. A method for operating an implantable medical device to detect a condition of a heart of a patient, comprising the steps of:
sensing an activity level of said patient;
sensing an acoustic energy;
producing acoustic signals indicative of heart sounds of the heart of said patient over predetermined periods of a cardiac cycle during successive cardiac cycles;
extracting a signal corresponding to a first heart sound (S1) from a sensed acoustic signal of a cardiac cycle;
calculating a relation between a first signal corresponding to a first heart sound (S1) in said first activity level range and a second signal corresponding to a first heart sound (S1) in a second predetermined activity level range; and
comparing said calculated relation with at least one reference value to detect said condition or a change of said condition.

28. (canceled)

29. The method according to claim 27, further comprising the step of:
calculating an energy value corresponding to an extracted signal corresponding to a first heart sound (S1); and wherein said step of comparing comprises the step of
comparing a present energy value corresponding to a first heart sound (S1) in a first activity level range with at least one reference value to detect said condition or a change of said condition.

30. The method according to claim 27, further comprising the step of
calculating said relation as a relation between an energy value corresponding to a first heart sound at said first activity level range and an energy value corresponding to a first heart sound at said second activity level range, and wherein said step of comparing comprises the step of
comparing said calculated relation with at least one reference value to detect said condition or a change of said condition.

31. The method according to claim 27, wherein the step of comparing comprises the step of:
comparing said calculated relation between an energy value corresponding to a first heart sound at said first activity level range and an energy value corresponding to a first heart sound at said second activity level range with at least one reference value to detect said condition or a change of said condition, wherein said condition is detected if the calculated relation is lower than a first predetermined reference value or higher than a second predetermined reference value.

32. The method according to claim 27, further comprising the step of storing signals and/or energy values corresponding to first heart sounds in said first and/or said second activity level.

33. The method according to claim 27, wherein each reference value corresponds to an extracted signal corresponding to a preceding first heart sound or an energy value corresponding to a preceding first heart sound.

34. The method according to claim 27, further comprising the step of calculating said at least one reference value as a mean value of a number of signals corresponding to preceding first heart sounds or as mean value of a number of energy values corresponding to preceding first heart sounds.

35. The method according to claim 27, wherein said step of comparing comprises the step of comparing a present signal corresponding to a first heart sound or a relation with a predetermined number of reference values or with reference values obtained during a predetermined period of time.

36. The method according to claim 27, further comprising the step of calculating each signal corresponding to a first heart sound (S1) at said first activity level as a mean value over a predetermined number of successive heart sound signals at said first activity level and each signal corresponding to a first heart sound (S1) at said second activity level as a mean value over a predetermined number of successive heart sound signals at said second activity level.

37. The method according to claim 27, further comprising the step of calculating each energy value corresponding to a first heart sound at said first activity level as a mean value over a predetermined number of successive energy values at said first activity level and an energy value corresponding to a first heart sound at said second activity level as a mean value over a predetermined number of successive energy values at said second activity level.

38. The method according to claim 27, wherein the step of extracting comprises the step of determining said first heart sound signal to be a part of said sensed signal having an amplitude above a predetermined amplitude level.

39. The method according to claim 27, wherein the step of calculating comprises the step of integrating a sensed signal during a first predetermined time window to obtain an energy value corresponding to said first heart sound signal.

40. The method according to claim 27, further comprising the steps of:
filtering out frequency components of said sensed signals outside a predetermined frequency range; and
rectifying said filtered signal to produce a signal containing only positive or zero values.

41. The method according to claim 27, further comprising the steps of
filtering out frequency components of said sensed signals outside a predetermined frequency range; and
performing a squaring procedure of said filtered signal to produce a signal containing only positive or zero values.

42. The method according to claim 40, further comprising the steps of:
identifying at least one local maximum point being coincident with a first heart sound signal; and
integrating said first heart sound signal in a predetermined time window comprising said at least one local maximum point.

43. The method according to claim 40, wherein said means signal processing circuit comprises:
selecting a part of said signal containing only positive or zero values above a predetermined threshold; and
integrating the part of the signal above said threshold, wherein an energy value corresponding to the first heart sound is obtained.

44. The method according to claim 27, detecting said condition or said change in said condition selected from the group consisting of arrhythmia, a conduction disorder, heart insufficiency, stress, and CHF.

45. The method according to claim 27, further comprising the steps of:
detecting at least one body position of said patient; and wherein
determining whether said patient is in said at least predetermined specific body position; and
using the body position information in said detection of said condition or change of said condition.

46. The method according to claim 45, further comprising the step of synchronizing a sensing session of said acoustic sensor with a determination that said patient is in a predetermined position.

47. The method according to claim 27, further comprising the step of synchronizing a sensing session of said acoustic sensor with a determination that said sensed activity level is below said predetermined activity level or that said sensed activity level is within a activity level range between a second activity level and a third activity level.

48. The method according to claim 27, further comprising the steps of
sensing a heart rate of said patient;
determining whether said heart rate is within a predetermined heart rate interval; and
if said patient is within said predetermined heart rate level range, calculating a relation between a first heart sound and a second heart sound for a heart cycle.

49. The method according to claim 27, comprising arranging said acoustic sensor in a lead connectable to said device.

50. The method according to claim 27, comprising arranging said acoustic sensor within a housing of said device.

51. The method according to claim 49, comprising placing said acoustic sensor is located in a lead in a left ventricle, in a right ventricle, or in a coronary vein of said patient.

52. The method according to claim 27, comprising selecting said acoustic sensor from the group consisting of accelerometer, pressure sensors and microphones.

53-54. (canceled)

55. A computer readable medium encoded with programming instructions for operating an implantable medical device to detect a condition of a heart of a patient, said programming instructions causing said medical device to:
sensed an activity level of said patient;
sensed an acoustic energy;
produced acoustic signals indicative of heart sounds of the heart of said patient over predetermined periods of a cardiac cycle during successive cardiac cycles;
extract a signal corresponding to a first heart sound (S1) from a sensed acoustic signal of a cardiac cycle;
calculate a relation between a first signal corresponding to a first heart sound (S1) in said first activity level range and a second signal corresponding to a first heart sound (S1) in a second predetermined activity level range; and
compare said calculated relation with at least one reference value to detect said condition or a change of said condition
